(19) United States
(12) Patent Application Publication
(10) Pub. No.: US 2013/0123621 A1
(43) Pub. Date: May 16, 2013

(54) DUAL CHAMBER IRRADIATION BALLOONS

(71) Applicants: John ISHAM, Houston, TX (US); Erik FRIJA, Houston, TX (US)

(72) Inventors: John ISHAM, Houston, TX (US); Erik FRIJA, Houston, TX (US)

(21) Appl. No.: 13/649,030

(22) Filed: Oct. 10, 2012

Related U.S. Application Data
(60) Provisional application No. 61/558,428, filed on Nov. 10, 2011.

(51) Int. Cl. A61M 25/10; A61N 5/10

(52) U.S. Cl. 600/435; 600/1

(57) ABSTRACT

Dual chamber, conforming, irradiation balloons with independent filling means can be filled with water or air in either chamber and allow fine control over dosimetry.

### PRIOR RELATED APPLICATIONS

This application claims priority to 61/558,428, filed Nov. 10, 2011, and expressly incorporated by reference herein in its entirety.

### FEDERALLY SPONSORED RESEARCH STATEMENT

Not applicable.

### JOINT RESEARCH AGREEMENT

Not applicable.

### FIELD OF THE INVENTION

The invention relates to irradiation balloons that are used to immobilize tissue or organ in place for radiation therapy.

### BACKGROUND OF THE INVENTION

“Radiation therapy,” as it is called in the US, is usually known as “radiation oncology” or “radiotherapy” in the UK, Canada and Australia. Radiation therapy, sometimes abbreviated as XRT, is the medical use of ionizing radiation, generally as part of cancer treatment to control malignant cells. Radiation oncology is the medical specialty concerned with prescribing radiation, and is distinct from radiology, which is the use of radiation in medical imaging and diagnosis. Radiation is often used as a therapeutic treatment, and can even be curative, but is also used for palliative treatment where cure is not possible and the aim is for local disease control or symptomatic relief. It is also common to combine radiation therapy with surgery, chemotherapy, hormone therapy, immunotherapy or some mixture of the four.

Radiation works by damaging the DNA of exposed tissue. It is believed that cancerous cells are more susceptible to death by this process, because many have turned off their DNA repair machinery during the process of becoming cancerous. To spare normal tissues from radiation damage, shaped radiation beams are aimed at the target from several angles of exposure to intersect and thus concentrate at the tumor. This provides a much larger dose at the target tumor site than in the surrounding, healthy tissue.

It is usually necessary, however, to include a margin of normal tissue around the tumor to allow for uncertainties in daily set-up and for tumor motion. Such uncertainties can be caused by internal movements (for example, respiration, bladder filling, peristaltic motions of the gastrointestinal tract, rectal gas and the like) and movement of external skin marks relative to the tumor position. We describe herein prostate irradiation as exemplary of the problem and proposed solutions, but the principles can be applied to other tissues and organs.

Researchers report the intrinsic motion of the prostate gland can be as much as 5 mm in the anterior to posterior direction due to rectal peristalsis. This has led to an additional 3 to 5 mm margin being added to the radiation field to account for prostate motion, along with 2 to 5 mm for setup error and dose buildup each, for a total margin of 10 to 15 mm to allow for the dose to reach 100% of the prescribed dose. If internal prostate motion is not addressed, it can lead to under-dosing of the target, and over-dosing of healthy surrounding tissues.

External patient positioning systems attempt to minimize anatomical variations by providing a secure and reproducible scaffolding, allowing the patient to comfortably maintain a relatively stable external position. However, neither CRT/IGRT/IMRT, nor the external positioning systems, can compensate for daily internal anatomical variations and organ movement due to breathing, rectal peristalsis, and rectal gas, which have been shown to be the major component of variation in target localization.

One way of minimizing the effects of internal motion is to compress the tissue with an inflatable balloon. However, most of the balloons on the market are non-conforming, thus lose their shape when overinflated or squeezed. Therefore, these balloons are less than ideal, with the prostate easily sliding off one side of the balloon or the other when in use.

RadiaDyne has provided an innovative solution to this problem, marketing a conforming rectal balloon that holds its shape even in the highly mobile environment of the rectum. This revolutionary new design has allowed the company to capture more than 90% of the prostate immobilizing rectal balloon market.

In more detail, the immobilizing balloon of US20080183202 and all related applications (incorporated by reference in their entirety) consists of three layers of material welded together at the edges, wherein the middle layer is also welded or glued to the upper layer. This weld or attachment point between the inner and upper layers provides a physical constraint against expansion on inflation or compression, and provides a groove or depression into which the prostate can be wedged during treatment.

US20080200872 (incorporated by reference in its entirety) provided a further improvement, allowing the distal surface to bulge on further inflation, thus further wedging the seminal vesicles in place and holding the balloon against expulsive forces. The distal bulge can be achieved in any number of ways, including making that portion of thinner material, making that portion of more elastomeric material, but more simply can be made by shifting the groove weld proximally, thus the greater amount of elastomeric material on the distal end will naturally stretch more. It is also possible to make a bulge or protrusion by welding on a e.g., semicircular portion of material (like the finger on a glove).

Application US2010014537 (incorporated by reference in its entirety) adds yet another improvement by including a rectal gas lumen so that gas can bypass the balloon while in use, thus minimizing both discomfort and internal motion. No prior balloons have ever thought to combine the balloon with a second lumen traversing the entirety of the balloon and allowing rectal gas to escape during treatment, and the clinical data available to date shows that this simple feature is surprisingly effective. Indeed, Ogino (2008) showed that prostate movement resulting from extreme distension of unstable rectal gas displaced the prostate up to 1.2 cm and concluded controlling rectal volume consistently is vital to reproducible RT treatment irrespective of IGRT utilization. See also Wootton L S et al., Effectiveness of a novel gas-release endorectal balloon in the removal of rectal gas for prostate proton radiation therapy, J Appl Clin Med Phys. 2012 Sep. 6; 13(5):3945 (2012) (“Thus gas-release balloon can effectively release gas, and may be able to improve clinical workflow by reducing the need for catheterization . . . . The modified ERB significantly decreased the overall frequency of fractions with gas present in any region by decreasing the frequency of fractions with gas present between the rectal balloon and the anterior rectal wall, the most common location of rectal gas. We conclude, therefore, that the gas-release ERB effectively removes rectal gas and should be used in patients receiving proton radiation therapy.”).

Application 61/551,745 showed another way to achieve the same conforming effect, wherein the balloon was internally welded to itself and/or the lumen. The principles are the same, a conforming shape is obtained by restraining the balloon in some way so as to prevent free expansion, and the restraint is typically a weld to either inner layer or structure or a weld to the opposite side of the balloon.

In all of the RadiaDyne applications discussed above, the balloon was a unitary balloon. Thus, the middle layer of balloon material had perforations or gaps so that the entire balloon consisted of a single fluid chamber and the entire device could be filled with a single lumen. This is shown in FIG. 1.

The RadiaDyne rectal balloons were significant improvements over the prior art non-conforming balloons, which were generally elastomeric and not physically constrained against bulging on compression, such the prostate could easily slide away. The RadiaDyne conforming balloons allowed reduction in margin surrounding the prostate, displacement of low lying bowel, reduction of dose related side effects, and the ability to escalate dose and increase the rate of local tumor control of prostate cancer. See e.g., S. BOTH, et al., Real-Time Study Of Prostate Intrafraction Motion During External Beam Radiotherapy With Daily Endorectal Balloon, Int J Radiat Oncol Biol Phys. (2011) 81(5):1302-9 (“Daily endorectal balloon [from RadiaDyne] consistently stabilizes the prostate, preventing clinically significant displacement (>5 mm).”)

However, there is always room for further improvements, and what is needed in the art are improvements in balloon design that allow further reductions in margins, improved dosimetry, and that also assist in imaging during treatment.

### BRIEF SUMMARY OF THE INVENTION

“Bolus” as used herein has been defined as “a specifically shaped material, which is usually tissue equivalent, that is normally placed either in direct contact with the patient's skin surface, close to the patient's skin surface, or inside a body cavity. This material is designed to provide extra scattering or energy degradation of the beam. Its purpose is usually to shape the dose distribution to conform to the target volume and/or to provide a more uniform dose inside the target volume.”

Recently, researchers have concluded that target coverage and rectal DVH indicators are preserved from prostate RT planning throughout the entire course of therapy if a daily, water filled, endorectal balloon (ERB) device is used.

Other clinicians, in contrast, have used an ERB filled with air to provide a constant, reproducible air-tissue interface at the posterior prostate wall. This approach has also been shown to provide a reproducible setup that allows for consistency between planning and delivery of radiotherapy for prostate cancer. It also takes advantage of the slight decrease in dose to the anterior rectal wall that occurs when photons traverse an air/tissue interface, that allows for a sparing of rectal mucosa while delivering full dose to the prostate.

Our solution to transient gas, prostate motion and the need to accurately position and dose the prostate, while avoid the rectal wall and other healthy tissue, was to design a dual chamber balloon, such that one chamber can be filled with water acting as a bolus and having a second chamber filled with air, and preferably also including a gas lumen for rectal gas to bypass the balloons and escape.

Providing a dual chamber ERB, with two independent filling means, provides the advantages of both systems. The dual chamber structure allows the radio-oncologist to choose which chamber is air or fluid filled, allowing improved dosimetry both during treatment planning and treatment delivery. The addition of bolus to the rectum provides a homogeneous target for treatment planning yielding reliable results with favorable DVHs, yet the gas balloon pushes the opposing rectal wall away from the target zone, and sparing it from radiation. Continuing the use of bolus through the treatment planning process removes the uncertainty inherent in the changing rectum, and thus ensuring each daily treatment is identical to the treatment plan. This duel balloon design can be effectively combined with a rectal gas lumen that conducts rectal gas past the balloon to the exterior, further improving the reproducible positioning and dosimetry.

### BRIEF DESCRIPTION OF THE DRAWINGS

A better understanding of the present invention can be obtained with the following detailed descriptions of the various disclosed embodiments in the drawings:

FIG. 1 is a cross section of a prior art endorectal balloon made with three layers, but a single fluid chamber, wherein a weld from the top layer to the middle layer creates a conforming shape that the prostate can be wedged into.

FIG. 2. is a cross section of a bolus rectal balloon having dual chambers that can be independently filled. This balloon retains the prior art dimple or groove into which the prostate can sit.

FIG. 3 is a top perspective view of the dual chamber bolus balloon, showing dimple or groove that is central in the horizontal axis, but shifted proximally in the longitudinal axis and provides more material distally, and thus allows a distal bulge when more inflated.

FIG. 4 is a cross section of a bolus rectal balloon having dual chambers that can be independently filled, as well as a gas lumen to carry gas out of the rectum, through the device, to the outside.

FIG. 5 is a cross section of a bolus rectal balloon having dual chambers that can be independently filled, a gas lumen to carry gas out of the rectum, and wherein the balloon filling means (e.g. lumen) do not traverse the balloon, but only enter the proximal end of the balloon via e.g., a low profile doghouse or some lumen fitting means.

FIG. 6 shows a horizontal cross section of a dual chamber bolus balloon, wherein a dimple is made by pinching a layer, and welding it to itself as well as to a central lumen or material layer.

FIG. 7 showing a horizontal cross section of a dual chamber bolus balloon, wherein the dimple is omitted altogether. The parts are otherwise the same as in FIG. 6.

FIG. 8 showing orientation of balloon axes as discussed herein.

### DETAILED DESCRIPTION OF THE INVENTION

The separation of the rectal balloon into two chambers, each of which can be independently filled, allows the radiation oncologist to fill, e.g., the chamber adjacent the prostate with water or saline or an aqueous contrast agent, and the other chamber with a gas, such as air. This dual chamber construction allows the radiation oncologist to more clearly target the prostate because the water bolus is tissue equivalent, and will slow and absorb radiation. Radiation will speed up in air, largely bypassing the air pocket. Thus, the physician can choose which chamber to fill with air or liquid such as water or saline, and thus effectively change and more finely control the dosimetry of surrounding tissues. Preferably, the balloon is also equipped with a gas lumen and is conforming—that is does not lose its shape when squeezed or overinflated.

The manufacture of conforming balloons is not a simple task. It is insufficient to merely make a mold of the desired shape, and pour latex over it. No matter the mold shape, the balloon's shape will be largely lost when overinflated or squeezed.

We have enabled a conforming balloon shape by using two, three or more layers of elastomeric film, with interior welds that restrict expansion of the balloon, and thus provide conforming depressions. Different shapes can also be achieved by making areas that need to be bigger (bulge more on inflation) with either a thinner material, a more elastic material, or by shifting placement of the weld, such that more material is available for expansion. Alternatively, a bulged portion can be welded to the surface of the balloon if a more extreme shape is needed. Alternatively, pre-shaping such as pressure/vacuum forming can result in a bulge. A large variety of shape balloon surfaces are possible by varying the placement and shape of the welds, and placement and shape of bulges.

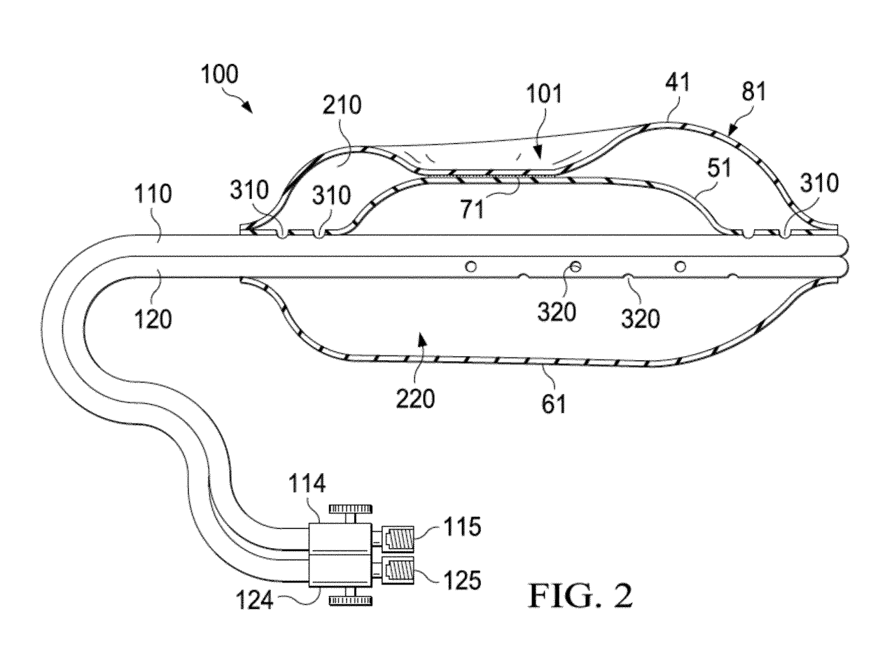

Welds can be to the top surface, the bottom surface or both. Such welds can be to a middle layer (e.g., a baffle layer), or to the lumen itself provided that the needed fluidic connections in the dual chambers are maintained. Thus, the balloon shown in FIG. 2 can easily be provided with a bottom dimple, by welding a portion of the bottom layer to the lumen or to an additional baffle layer. Where greater distance from the lumen is needed, a spacer or baffle layer can be added, or the balloon layer can be pinched and welded to itself, such that the weld to the lumen is further from the layer surface.

We have shown two chambers herein, but of course the device could be provided with three or more chambers, by merely expanding on the principals herein. However, the simplest design is preferred, as generally being the least expensive to manufacture.

We exemplify the dual chamber bolus balloon herein with a shape that conforms to a rectal space, with a groove or depression for the prostate to wedged into (through the rectal wall), but the design principals can be applied to other balloon shapes and thus used in other body cavities.

The dual chamber rectal balloon can also be advantageously provided with a third lumen that traverses the entire length of the balloon, protruding past the distal end, and provides a passageway for the escape of rectal gas. Ideally, such lumen has a smooth, soft tip preferably with multiple holes for gas entry, and is positioned adjacent the other two lumens. However, other positions and shapes are possible.

When a gas lumen is provided, the other two lumens need not traverse the balloon, but can merely emerge from the proximal end of the balloon via a connector means, such as a low profile inlet fitmet, which is well known in the art. This minimizes the device cross section, making it smaller and more comfortable to the patient on insertion and removal. Nested catheters can also be used, or multiple catheters bonded together, or a large catheter can be divided into two or three spaces, wherein considerations of patient comfort and cost will drive these design choices.

The dual chamber bolus balloon can also comprise radio-opaque markers that can be used in imaging for accurate placement of the balloon. Radio-opaque markers can be letters indicating top (T) or right (R) and left (L) sides of the balloon, or numbers or any other shape, and can be particularly advantageous for those balloons whose shape is not radially symmetrical. A marker can also be placed on the very tip of the gas lumen, if included therein. Many radiopaque materials are known, and include palladium, platinum, gold, iridium, rhenium and rhodium, silver, tin, tantalum, tungsten and alloys thereof, tungsten being preferred.

The dual chamber bolus balloon can also comprise passive radiation sensors, such as is used in radiation badges. Electronic radiation sensors can also be used, but may contribute significantly to expense, and may be less appropriate for a disposable balloon. Motion sensors, temperature sensors, and other types of sensors can also be included thereon.

Preferably, the dual chamber rectal balloon is provided with locking stopper that serves to prevent the balloon from sliding further into the rectum, which it is prone to do without such stopper. The stopper has an upper portion, generally smoothly rounded or hemispherical, which fits snugly against the anus, and a hole or groove, through which the lumen(s) is/are threaded. Other shapes may be used for other body cavities, and the stopper may also be optional for most cavities.

A lower locking portion of the stopper snap locks against the lumen without blocking fluid entry, and preferable has interior fins or ridges lining its hole that engage the lumen, and prevent sliding, as a locking mechanism without such ridges is prone to do. Another means of making a locking stopper is to line the interior of the hole or groove through which the lumens are threaded with a tacky material, so that friction locks the stopper in place. Another embodiment has a conical or other shaped interior opening that compresses against the tube outer diameter, but not so much as to block same. A hinge on the locking portion allows the lock to be opened, and the lock snap fits shut.

The details of the locking mechanism can be as shown in US2010145379 or WO2010141024, both incorporated herein by reference herein in their entirety. The upper portion of the locking stopper of US2010145379 has a groove reaching to the central hole, so that the stopper need not be threaded over the lumen, but this groove can be replaced with just a hole and thus prevent stopper loss once the valves and luer lock are added to the end of the lumen. Of course, the central hole or groove is not necessarily round as shown in US2010145379, especially if two or three lumens are welded together, but should reflect the cross section of the lumen(s).

Preferably the balloon material is an elastomeric polymer. Thermoplastic material with a specific vicat temperature (point at which the resin softens) can be used to promote additional anatomic conformance at body temperature.

The balloon is preferably made of thermoplastic elastomers (TPE), especially thermoplastic polyurethane. Other balloon fabrication materials include latex, polyethylene (PE), polypropylene (PP), silicone, vinyl, polyvinyl chloride (PVC), low density polyethylene (LDPE), polyvinylidene chloride (PVDC), linear low density polyethylene (LLDPE), polyisobutene (PIB), and poly[ethylene-vinylacetate] (EVA) copolymers, nitrile, neoprene, and the like. It is also possible to use a laminar plastic, having more than one layer, e.g., a tougher interior layer and a biocompatible or slippery outer layer.

The ideal material is a biocompatible material that has a durometer of less than 80-100 Shore A (ASTM D2240 or ISO 868), a tensile strength of at least 3000 psi (ISO 527-3 or ASTM D882-02), a 100% modulus of 500-1000 psi (ASTM D412), an elongation at break of at least 300% (ASTM D412), and that is air tight even under 150% stretch. In some applications, the material should also be sterilizable, but this is not needed for a rectal balloon. Translucent or transparent materials are also preferred.

One preferred material is an ether based thermoplastic polyurethane of 70-100 Shore A, preferably 80-90 Shore A Durometer hardness, and a thickness of 0.003-0.015 inch. This material shows outstanding abrasion and wear resistance, superior toughness and durability, yet allows ease of processing and manufacturing flexibility. It also has a nice surface feel that allows the balloon to easily slide into the body. Additionally, polyurethane is manufactured without the use of plasticizers, which means it will retain its original performance characteristics for longer time periods. Not having plasticizers also means it will not leach out hazardous compounds, which is of particular importance for medical uses.

The bolus chamber balloon can be made in three or more layers that are welded to form the correct shape. By “weld” herein we mean any method of attaching two layers of polymer film together. Thus, the welds or attachment points can be heat welded, RF welded, ultrasound welded, glued, solvent welded, hot gas welded, freehand welded, speed tip welded, extrusion welded, contact welded, hot plate welded, high frequency welded, injection welded, friction welded, spin welded, laser welded, impulse welded or any other means known in the art.

By “centrally” weld we do not imply an exact center, but instead use this term more generally to distinguish a central weld from welds that may be made at one or more edges of a balloon to form a fluid-tight chamber. Thus, a central weld can be anywhere inside the edges, and can even reach to an edge if desired.

We have built prototype models using three layers of flat film welded at the edges etc. It is possible, however, to build a conforming balloon with a molded balloon. The conforming depression is achieved by pinching one balloon centrally and welding the pinch shut, and then welding the inside end of the pinch to the shaft or lumen. The second balloon is then welded to the first balloon, with the shaft therebetween. As above, the two balloons can be provided with independent fluid filling means via a low profile doghouse, or the central shaft can include independent fluid passageways, and a third gas lumen for rectal applications.

Combinations of the above manufacturing techniques can also be used.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise,” “have,” “include,” and “contain” (and their variants) are open-ended linking verbs and allow the addition of other elements when used in a claim. The term “consisting of is a closed linking verb, and does not allow the addition of other elements.

The term “consisting essentially of occupies a middle ground, allowing non-material elements to be added. In this case, these would be elements such as marking indicia, radio-opaque markers, a stopper, packaging, instructions for use, labels, and the like.

The following abbreviations are used herein:

| | |
|---|---|
| CRT | conformal radiation therapy |
| CT | computer tomography |
| DVH | dose-volume histogram |
| ERB | endorectal balloon |
| IGRT | image guided radio therapy |
| IMRT | intensity-modulated radiation therapy |
| MRI | magnetic resonance imaging |
| PET | position emission tomography |
| RD | rectal diameter |
| RV | rectal volume |
| XRT | radiation therapy |

### EXAMPLE 1

### Prior Art

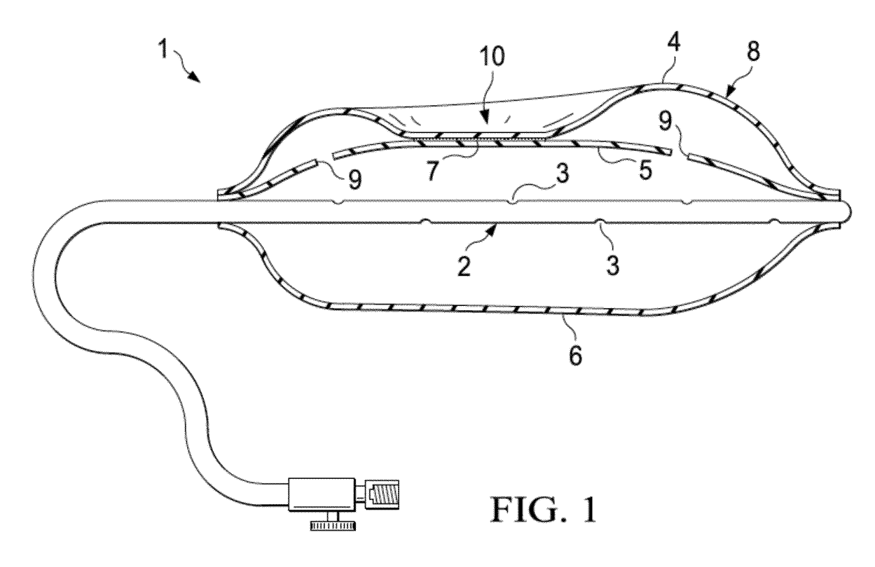

FIG. 1 shows a cross section of the prior art rectal balloon 1 with lumen 2 having offset holes 3 for fluidic communication with the interior of the balloon. The use of a plurality offset holes is generally preferred because it helps to prevent inadvertent hole blockage, e.g., by the balloon material or the rectal walls.

The balloon comprises a top layer 4, a middle layer 5, and a bottom layer 6, which are welded together along the edges (not shown), and also affixed to the lumen, in this case at both the distal and proximal ends. The top layer 4 is welded 7 to the middle layer 5 along the central line of the balloon, but shifted proximately, so that the distal portion of the balloon bulges 8 more than the proximal portion on hyperinflation. The middle layer also has holes or gaps 9 so that the balloon comprises only a single fluid chamber. The balloon filling means (typically a lumen, stock cock and luer connector) are not shown in this figure, but are typical in the art.

The weld 7 of top layer 4 to middle layer 5 provides a groove 10 (or dimple, indent, depression) having some depth into which the prostate can be wedged. The physical coupling of the middle layer to the top layer provides a physical restraint against expansion or stretching, and thus the balloon is conforming—that is it holds its shape even in the highly mobile environment of the rectum. In this instance, the balloon was manufactured inside out and then inverted so as to prevent contact between the stiffer welds and the rectal tissue.

### EXAMPLE 2

### Dual Chamber Bolus Balloon

Ether based thermoplastic polyurethane pellets were obtained and melt, blown, or extruded film of 0.003-0.015 inch thick was prepared.

Two lumens with appropriately spaced holes were bound together, and fitted with closable means for injecting fluid. If desired, a third gas lumen can be added.

The films are cut to shape, generally ovoid or football shaped. The top layer is welded to the middle layer, wherever a depression is desired. The three layers are then welded together along the edges, and a small lumen opening left at each end. The lumens are threaded therethrough, and the middle layer is welded to the top lumen at the extreme ends, and holes provided in said weld such that said top lumen is in fluid communication with the upper chamber. The ends of the top and bottom layers are also welded to the lumens. The chambers can also be inflated by use of a multi-lumen, nested catheter. The tip of the balloon can also be made of a different durometer material and bonded onto the catheter using welding or UV bonding.

The completed dual chamber bolus balloon is shown in FIG. 2-4.

FIG. 2 shows a cross section of a dual chamber bolus balloon 100 comprising two or three fluid passageways extending therethrough (two shown in this embodiment). The first fluid passageway or tube or lumen 110 connects to the first balloon 210, the second lumen 120 connects to the second balloon 220. Holes 310 and 320 connect to the first balloon chamber 210 and second balloon chamber 220 respectively. Thus, first lumen 110 fluidly connects with first balloon 210 and second lumen 120 fluidly connects with second balloon 220.

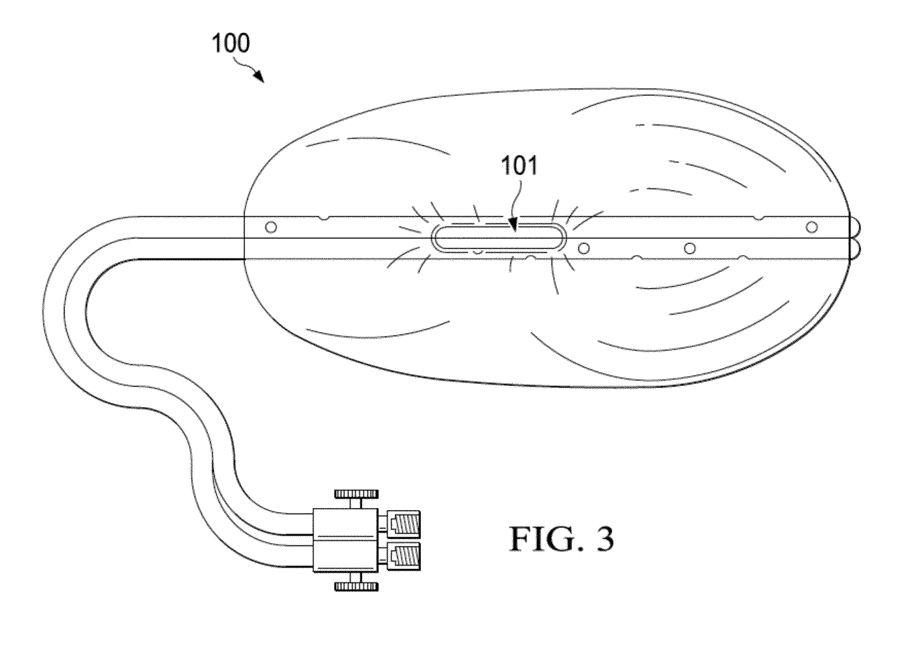

The same balloon is shown inflated and in perspective in FIG. 3, and the generally flat upper surface with central depression or groove 101 can be seen. This is made by welding 71 upper layer 41 to middle layer 51 in a central location, or as needed to reflect the desired shape. Thus, top layer and middle layer make up one balloon chamber 210 and middle and bottom layer 61 make up the other chamber 220.

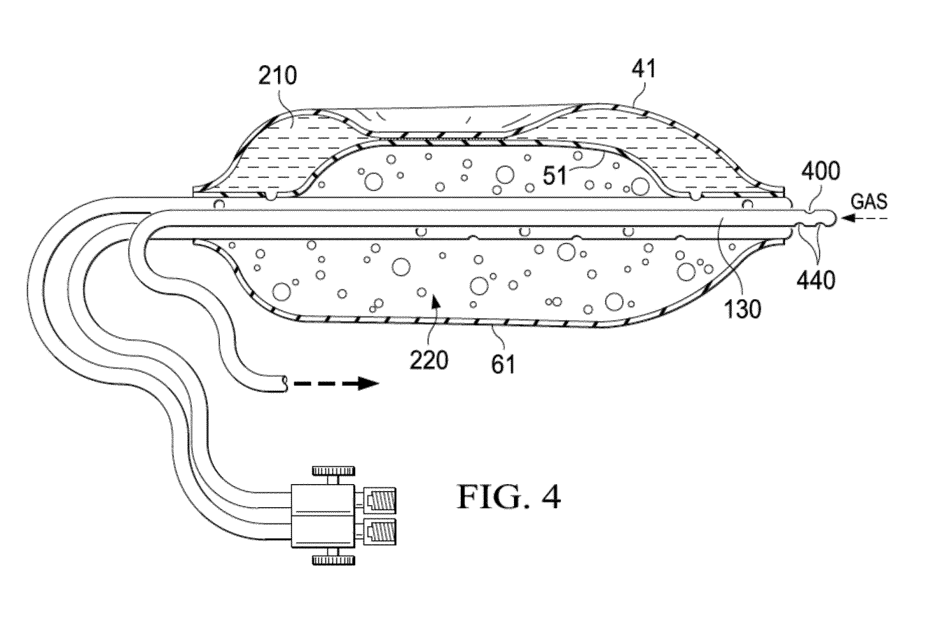

FIG. 4 shows a variation with a third gas lumen 130, which connects with the distal-most exterior of the dual balloon apparatus 100 with the proximal-most exterior. The gas lumen has a soft flexible tip 400 with holes 440. The proximal end of the gas lumen can be either open as shown here or reversibly closable.

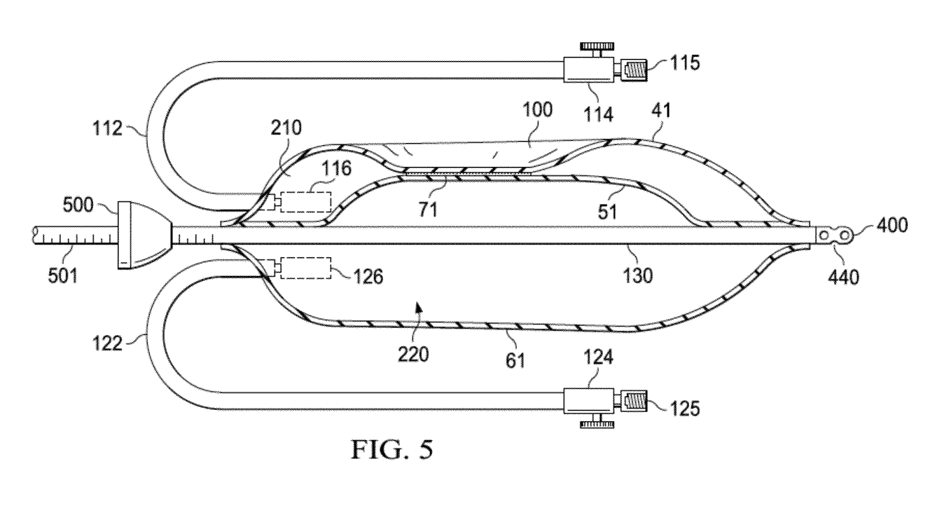

FIG. 5 shows a variation wherein only the gas lumen 130 traverses the balloon, and the two fluid filling means connect to the proximal end of the balloon, e.g., via doghouse on the surface on the surface of the two balloons.

As in the prior art balloons, the dual chamber balloon 100 of FIG. 2 is made in three layers—top layer 41, middle layer 51, and bottom layer 61. Top layer 41 is welded 71 to middle layer 51, creating a dimple or groove 101 and optional distal bulge 81. Each layer is also welded to the pair of lumens 110, 120, preferably at both the distal and proximal edges, but middle layer 51 is also welded to the lumen 110 a short distance interior to the other welds, yet traversing the lumen to meet the other welds, and holes 310 provided in this short portion so that the top chamber 210 can be in fluid communication with first lumen 110. The short distance is shown exaggerated herein for clarity.

The middle layer weld to the lumen can either have a gapped portion to fit over the holes, yet provide an air tight fit, or can be welded to the lumen, and the hole provided through such weld, as desired or as easiest to manufacture. The exact means of making the two chambers airtight will of course vary with the design of the fluid entry means.

We have shown both lumens travelling all the way to the distal end of the balloon in FIG. 2 so that there can be some central support structure allowing for insertion of the balloon into a body cavity. However, one of the lumens can be omitted, and the other chamber merely fitted with fluid entry means, such as a low profile lumen fitting as in 116/126 of FIG. 5, to which a short lumen and valve means are inserted. Alternatively, if a gas lumen is provided, both chamber lumens can be limited to a short portion of lumen protruding from the proximal end of each chamber. This is shown in FIG. 5.

Preferably, a lumen that traverses the balloon, e.g., the gas lumen in FIG. 5 or at least one lumen in FIG. 2, is a semi-rigid plastic or other material that provides enough stiffness for the device to be inserted into the rectum, yet still allows some flex. At the same time, the end of the lumen connected to air intake means should be much more flexible in preferred embodiments, so that that entire device is not moved when the medical professional is filling the balloons. A simple connector can be used to connect lumens of differing flexibility, such that inside the balloon the lumen is stiffer, and proximately it is more flexible, or the gas lumen alone can provide the semi-rigid shaft needs, whilst the fluid injector lumens are quite flexible.

FIG. 3 shows the dual chamber bolus balloon in perspective, which clearly illustrates the central groove or dimple, which is oval or linear or rectangular, and into which the prostate can be wedged while in use. This rectal shape is exemplary only and using the central weld principal herein, dimples can be provided wherever needed. Further, this application expressly contemplates a dual chamber balloon that lacks the central weld and dimple altogether, as shown in FIG. 7.

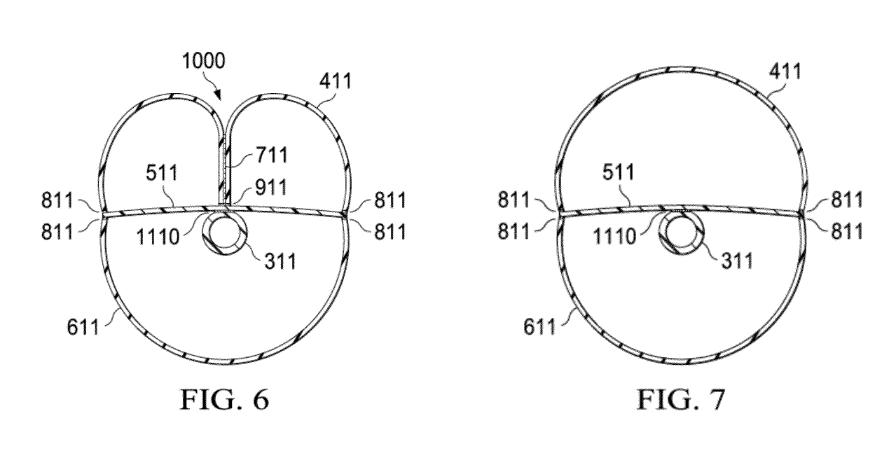

FIG. 6 shows a balloon wherein the dimple 1000 is created by pinching the upper layer 411 and welding the pinch 711. The end of the pinch is then welded to the middle layer 511, which is welded 1110 to the gas lumen 311. The lumen can either be above the middle layer (not shown) or below the middle layer as shown, and the welds adjusted accordingly. The bottom layer 611, middle layer 511 and top layer 411 are welded together at the edges 811 to form the two balloon chambers. Not shown in this horizontal cross section or in FIG. 7 are the lumens that fluidly communicate with the balloon chambers.

When packaged, the dual chamber balloons are vacuumed against the shaft or lumen, and folded tightly against the lumen for minimal profile. The empty balloon is then inserted into the patient with the groove positioned adjacent the prostate, as indicated by indicia 501 on the lumen. The lockable stopper 500 is closed at the desired position, preventing the balloon from being pulled further into the rectum on inflation. The chambers are filled by physician choice with water or air, usually via a syringe so that the volume of fill is reproducible. The balloon can be imaged during use, for example with the use of radiopaque markers, and this also ensures reproducibility. When positioned, the radiation treatment can then proceed.

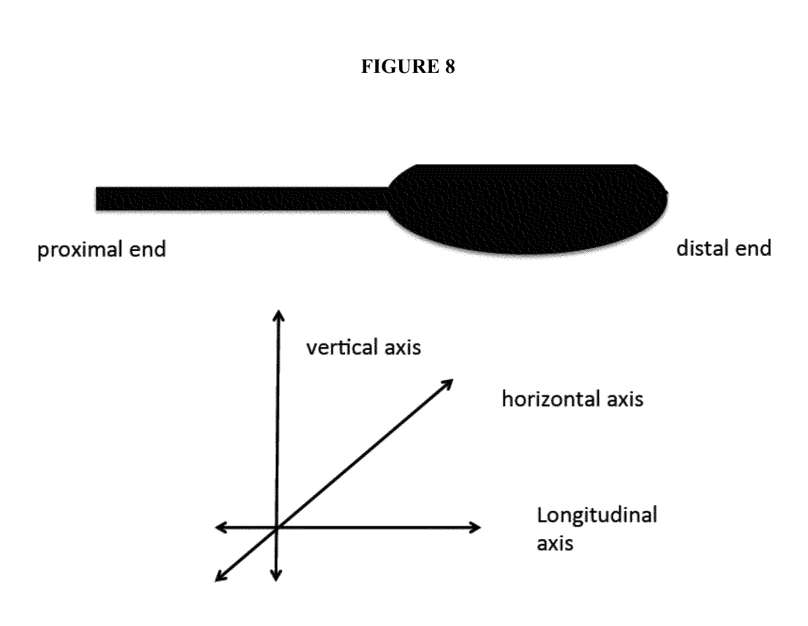

The term distal as used herein is the end of the balloon inserted into the body cavity, while proximal is opposite thereto. The terms top and bottom are in reference to the figures only, and do not necessarily imply an orientation on usage. The length of balloon plus lumen is the longitudinal axis, while a horizontal axis and vertical axis cross the longitudinal axis, and the cross sections in the figures are shown across the vertical axis. See FIG. 8 showing these axes.

The foregoing disclosure and description of the invention are illustrative and explanatory thereof, and various changes in the details of the illustrated apparatus and system, and the construction and method of operation may be made without departing from the spirit of the invention.

The following references are incorporated by reference in their entirety herein.

61/551,745

US20080183202

US20080200872

US2010014537

Ogino, I., et al., Reduction of Prostate motion by removal of gas in rectum during radiotherapy, Int. J. Radiation Oncology Bio. Phys. 72 (2), 456-466 (2008).

Both, S., et al., Real-Time Study Of Prostate Intrafraction Motion During External Beam Radiotherapy With Daily Endorectal Balloon, Int J Radiat Oncol Biol Phys. 81(5):1302-9(2011).

Wootton L. S. et al., Effectiveness of a novel gas-release endorectal balloon in the removal of rectal gas for prostate proton radiation therapy, J Appl Clin Med Phys. 2012 Sep. 6; 13(5):3945 (2012), available online at http://www.jacmp.org/index.php/jacmp/article/view/3945/2645.

What is claimed is:

1. A dual chamber bolus balloon, said balloon comprising:
a) a distal end and a proximal end;
b) a first airtight chamber and a second airtight chamber adjacent each other;
c) said first chamber being fluidly connected to a first means for providing a first fluid to said first chamber;
d) said second chamber fluidly connected to a second means for providing a second fluid to said second chamber;
e) said first chamber being made of a top layer and a middle layer; and
f) said second chamber being made of said middle layer and a bottom layer.

2. The dual chamber bolus balloon of claim 1, further having at least one central weld between said top layer and said middle layer, said weld providing a depression in said first chamber when in an inflated condition.

3. The dual chamber bolus balloon of claim 1, further comprising a gas lumen passing beyond said distal end to beyond said proximal end, and allowing gas to pass therethrough.

4. The dual chamber bolus balloon of claim 3, said gas lumen comprising a flexible tip with a plurality of holes therein.

5. The dual chamber bolus balloon of claim 1, further comprising at least one radio-opaque marker.

6. The dual chamber bolus balloon of claim 4, said gas lumen comprising a radio-opaque marker at its proximal tip.

7. The dual chamber bolus balloon of claim 1, further comprising at least one sensor.

8. The dual chamber bolus balloon of claim 1, further comprising at least one radiation sensor.

9. The dual chamber bolus balloon of claim 1, further comprising at least one motion sensor.

10. The dual chamber bolus balloon of claim 1, wherein each of said first and second means for providing a first and second fluid comprises a lumen with stopcock and luer lock, said lumen having at least one opening into respective first and second chambers.

11. The dual chamber bolus balloon of claim 4, wherein said at least one opening is a plurality of offset openings.

12. The dual chamber bolus balloon of claim 1, wherein at least one of said lumens protrudes from the proximal end and has distance indicia thereon.

13. The dual chamber bolus balloon of claim 4, wherein at least one of said lumens protrudes from the proximal end and has distance indicia thereon.

14. The dual chamber bolus balloon of claim 1, wherein said top, middle and bottom layers are welded together around an edge of each layer, and also welded to said lumens at said distal end and said proximal end.

15. The dual chamber bolus balloon of claim 4, wherein said top, middle and bottom layers are welded together around an edge of each layer, and also welded to said lumens at said distal end and said proximal end.

16. The dual chamber bolus balloon of claim 1, wherein said layers comprise thermoplastic polyurethane.

17. The dual chamber bolus balloon of claim 4, further comprising a lockable stopper slidingly positioned over said gas lumen at a proximal end of said balloon, said lockable stopper having a smoothly curved distal surface.

18. The dual chamber bolus balloon of claim 1, wherein said layers comprise thermoplastic polyurethane having a 80-100 Shore A durometer, a tensile strength of at least 3000 psi, a 100% modulus of 500-1000 psi, and elongation before break of at least 300%.

19. The dual chamber bolus balloon of claim 4, wherein said layers comprise thermoplastic polyurethane having a 80-100 Shore A durometer, a tensile strength of at least 3000 psi, a 100% modulus of 500-1000 psi, and elongation before break of at least 300%.

20. A dual chamber irradiation balloon, comprising:
a) a first airtight chamber having a lumen and valve means in fluid communication said first chamber;
b) a second airtight chamber having a lumen and valve means in fluid communication said second chamber;
c) a flexible gas lumen passing from a first end of said balloon to an opposite end of said balloon, and providing a gas passageway all the way through said balloon;
d) said first and second chambers being adjacent and being formed of a top layer, a middle layer and a bottom layer, said middle layer being shared between said first and second chambers;
e) said layers being welded together at edges and to said lumens at least at a proximal end of each of said first and second chambers; and
f) said top layer centrally welded to said middle layer or to said gas lumen to form a conforming depression in said first chamber when inflated.

21. The dual chamber irradiation balloon of claim 20, further comprising distance markings at least one of said lumens or said gas lumen.

22. The dual chamber irradiation balloon of claim 20, said lumens comprising a plurality of offset holes in communication with first and second chambers, respectively.

23. The dual chamber irradiation balloon of claim 20, said gas lumen having a soft tip with a plurality of offset holes therein.

24. The dual chamber irradiation balloon of claim 20, further comprising at least one radiopaque marking on a surface of said balloon or on a lumen or on a distal tip of a lumen.

25. The dual chamber irradiation balloon of claim 20, further comprising at least one radiation sensor on a surface of said balloon or on said lumen or on said gas lumen or at said weld.

26. The dual chamber irradiation balloon of claim 20, which is shaped to fit a male rectum such that a prostate can be wedged into said depression in said first chamber when inflated.

27. The dual chamber irradiation balloon of claim 20, which includes a lockable stopper on one or more of said lumens or said gas lumen.

28. An improved radiation balloon for constraining a body tissue for external beam radiation therapy, said radiation balloon having an inflatable chamber and a fluid filling means for said chamber, the improvement comprising at least two conjoined inflation chambers each with an independent fluid filling means, such that one chamber can be filled with air and the other chamber filled with a tissue equivalent fluid or aqueous contrast agent.

29. An improved radiation balloon for constraining a body tissue for external beam radiation therapy, said radiation balloon having an inflatable chamber and a fluid filling means for said chamber, the improvement comprising at least two conjoined inflation chambers made with three layers of material welded together along each edge, each inflation chamber having an independent fluid filling means, such that one chamber can be filled with air and the other chamber filled with a tissue equivalent fluid or aqueous contrast agent.

30. An improved radiation balloon for constraining a body tissue for external beam radiation therapy, said radiation balloon having an inflatable chamber and a fluid filling means for said chamber, the improvement comprising at least two conjoined inflation chambers made with top, middle and bottom layers of material welded together at edges of said layers, wherein the top layer is centrally welded to the middle layer to create a dimple when said balloons are inflated, each inflation chamber having an independent fluid filling means, such that one chamber can be filled with air and the other chamber filled with a tissue equivalent fluid or aqueous contrast agent.

31. A method of radiation treatment, comprising inserting the dual chamber bolus balloon of claim 1, 20, 28, 29 or 30 into a body cavity of a patient, filling one chamber with air and filling the other chamber with an aqueous solution, and providing radiation therapy to said patient at or near said body cavity.
